(19) United States
(12) Patent Application Publication
(10) Pub. No.: US 2023/0257822 A1
(43) Pub. Date: Aug. 17, 2023

(54) METHODS FOR DETECTING TISSUE DAMAGE, GRAFT VERSUS HOST DISEASE, AND INFECTIONS USING CELL-FREE DNA PROFILING

(71) Applicant: CORNELL UNIVERSITY, Ithaca, NY (US)

(72) Inventors: Iwijn DE VLAMINCK, Ithaca, NY (US); Alexandre Pellan CHENG, Ithaca, NY (US); Matthew Pellan CHENG, Ithaca, NY (US); Francisco Miguel MARTY, Ithaca, NY (US); Jerome RITZ, Ithaca, NY (US)

(21) Appl. No.: 17/920,931

(22) PCT Filed: Apr. 23, 2021
(86) PCT No.: PCT/US2021/028820

§ 371 Date: Oct. 24, 2022

Related U.S. Application Data
(60) Provisional application No. 63/015,095, filed on Apr. 24, 2020.

(51) Int. Cl. C12Q 1/6886; C12Q 1/6888

(52) U.S. Cl. CPC C12Q 1/6886; C12Q 1/6888; C12Q 2600/154

(57) ABSTRACT

The present disclosure is directed to novel methods for detecting tissue damage, graft-versus-host disease (GVHD), microbial infections, presence of a tumor, and loss of engraftment in a subject using cell-free DNA (cfDNA) profiling. The methods of the disclosure are in part based on the recognition that damaged tissues, microbes during an infection, tumors, and donor cells (e.g., in a hematopoietic cell transplantation) shed small fragments of cfDNA into blood circulation.

### CROSS REFERENCE TO RELATED APPLICATION

This application claims the benefit of priority from U.S. Provisional Application No. 63/015,095, filed Apr. 24, 2020, the entire contents of which are incorporated herein by reference.

### STATEMENT REGARDING FEDERALLY SPONSORED RESEARCH OR DEVELOPMENT

This invention was made with government support under Grant Nos. DP2AI138242 and R01AI146165, awarded by the National Institutes of Health. The government has certain rights in the invention.

### BACKGROUND

Allogeneic hematopoietic cell transplantation (HCT) provides effective treatment for hematologic malignancies and immune disorders. Monitoring for immune complications and infection is a critical component of post-HCT therapy, however, current diagnostic options are limited.

More than 30,000 patients undergo allogeneic hematopoietic cell transplants (HCT) worldwide each year for treatment of a variety of malignant and nonmalignant hematologic diseases. However, immune related complications occur frequently after HCT. Up to 50% of patients experience graft-versus-host disease (GVHD) in the first year after transplantation. GVHD occurs when donor immune cells attack the patient's own tissues. Early and accurate diagnosis of GVHD is critical to inform treatment decisions and to prevent serious long-term complications, including organ failure and death. Unfortunately, there are few, noninvasive diagnostic options that reliably identify patients very early after onset of GVHD symptoms. In current clinical practice, diagnosis of GVHD relies almost entirely on clinical criteria and often requires confirmation with invasive procedures, such as a biopsy of the gastrointestinal tract, skin, or liver.

Small fragments of cell-free DNA (cfDNA) circulate in blood. In the absence of disease, cfDNA originates primarily from apoptosis of cells of the hematopoietic lineage. During disease, a significant proportion of cfDNA can be derived from affected tissues. In solid-organ transplantation (SOT), it has been shown that transplant donor derived cfDNA in the blood is a quantitative noninvasive marker of solid organ transplant injury.

### SUMMARY OF THE DISCLOSURE

An aspect of this disclosure is directed to a method for detecting tissue damage in a subject comprising obtaining cfDNA molecules from a biological sample from the subject; determining the profiles of an epigenetic marker within the cfDNA molecules, wherein the epigenetic marker displays tissue-specific profiles; identifying the tissues of origin of the cfDNA molecules based on the profiles determined; and measuring the level of cfDNA molecules from an identified tissue of origin, wherein (i) the level or (ii) an increased level of cfDNA molecules from said identified tissue of origin as compared to a control level, is indicative of damage in said identified tissue of origin.

In some embodiments, the epigenetic marker is selected from the group consisting of a DNA modification, a histone modification, and nucleosome positioning. In some embodiments, the DNA modification is DNA methylation or DNA hydroxymethylation. In some embodiments, the histone modification is selected from the group consisting of acetylation, methylation, phosphorylation, ubiquitylation, GlcNAcylation, citrullination, krotonilation, and isomerization.

In some embodiments, the step of determining the profiles of the epigenetic marker comprises determining the sequences of the cfDNA molecules.

In some embodiments, the profile of DNA methylation is determined by bisulfite treatment or enzymatic DNA methylation analysis. In some embodiments, the profile of DNA hydroxymethylation is determined by a pull-down assay, a selective labeling assay, or an oxidative bisulfite sequencing assay. In some embodiments, the profile of histone modification is detected by a pull-down assay. In some embodiments, the nucleosome positioning is determined by a nucleosome positioning assay. In some embodiments, the determining the profiles of the epigenetic marker is achieved without determining the sequences of the cfDNA molecules.

In some embodiments, prior to determining, a library of cfDNA molecules is prepared using single-stranded DNA (ssDNA) library preparation method.

In some embodiments, the determining is achieved by a PCR assay selected from quantitative PCR (qPCR) and digital droplet PCR (ddPCR).

In some embodiments, the assay comprises amplifying cfDNA molecules from regions of the genome that have specific epigenetic markers.

In some embodiments, the subject has undergone hematopoietic cell transplantation (HCT).

In some embodiments, the biological sample is a blood or a serum sample.

In some embodiments, the biological sample is obtained from the subject about 15 days, about 30 days, about 45 days, about 60 days or about 90 days after the HCT.

In some embodiments, the control level is (i) the level of cfDNA molecules in a sample from the subject prior to HCT, or (ii) the level of cfDNA molecules in a sample from a subject who has undergone HCT but who has not had graft-versus-host disease (GVHD).

In some embodiments, the tissues of origin comprise a solid organ. In some embodiments, the solid organ is an organ selected from kidney, liver, spleen, and pancreas. In some embodiments, the tissues of origin comprise a tumor. In some embodiments, the cfDNA molecules are from one or more organs selected from skin, heart, kidney, liver, lungs, stomach, bladder or pancreas.

In some embodiments, when there is a tissue damage in the subject, the method further comprises treating the subject with a therapy to ameliorate a damaged tissue in the subject. In some embodiments, the therapy comprises administration of an immunoregulatory agent to the subject.

In some embodiments, the damaged tissue is selected from skin, heart, kidney, liver, lungs, stomach, small intestine, large intestine, bladder, and pancreas, and immunoregulatory agent is selected based on tissue injury pattern.

In some embodiments, detecting tissue damage at about 30 days post-HCT is indicative of organ rejection or a risk of developing organ rejection.

In some embodiments, the tissue damage is indicative of graft-versus-host disease (GVHD). In some embodiments, the method further comprises treating the subject with an immunoregulatory agent when there is GVHD in the subject.

In some embodiments, the tissue damage is indicative of a microbial infection. In some embodiments, the method further comprises treating the subject with an antibiotic or an antiviral drug.

In some embodiments, the tissue damage is indicative of drug toxicity.

Another aspect of the disclosure is directed to a method for monitoring a subject who has undergone hematopoietic cell transplantation (HCT) comprising obtaining cfDNA molecules from a biological sample from the subject; determining the profiles of an epigenetic marker within the cfDNA molecules, wherein the epigenetic marker displays tissue-specific profiles; identifying the tissues of origin of the cfDNA molecules based on the profiles determined; and measuring the level of cfDNA molecules from an identified tissue of origin, wherein an increased level of cfDNA molecules from said identified tissue of origin as compared to a control level is indicative of graft-versus-host disease.

In some embodiments, the epigenetic marker is selected from the group consisting of a DNA modification, a histone modification, and nucleosome positioning. In some embodiments, the DNA modification is DNA methylation or DNA hydroxymethylation. In some embodiments, the histone modification is selected from the group consisting of acetylation, methylation, phosphorylation, ubiquitylation, GlcNAcylation, citrullination, krotonilation, and isomerization.

In some embodiments, the determining the profiles of the epigenetic marker comprises determining the sequences of the cfDNA molecules.

In some embodiments, the profile of DNA methylation is determined by bisulfite treatment or enzymatic DNA methylation analysis. In some embodiments, the profile of DNA hydroxymethylation is determined by a pull-down assay, a selective labeling assay, or an oxidative bisulfite sequencing assay. In some embodiments, the profile of histone modification is detected by a pull-down assay. In some embodiments, the nucleosome positioning is determined by a nucleosome positioning assay. In some embodiments, the determining the profiles of the epigenetic marker is achieved without determining the sequences of the cfDNA molecules.

In some embodiments, prior to determining, a library of cfDNA molecules is prepared using single-stranded DNA (ssDNA) library preparation method.

In some embodiments, the determining is achieved by a PCR assay selected from quantitative PCR (qPCR) and digital droplet PCR (ddPCR).

In some embodiments, the assay comprises amplifying cfDNA molecules from regions of the genome that have specific epigenetic markers.

In some embodiments, the subject has undergone hematopoietic cell transplantation (HCT).

In some embodiments, the biological sample is a blood or a serum sample.

In some embodiments, the biological sample is obtained from the subject about 15 days, about 30 days, about 45 days, about 60 days or about 90 days after the HCT.

In some embodiments, the control level is (i) the level of cfDNA molecules in a sample from the subject prior to HCT, or (ii) the level of cfDNA molecules in a sample from a subject who has undergone HCT but who has not had graft-versus-host disease (GVHD).

In some embodiments, the tissues of origin comprise a solid organ. In some embodiments, the solid organ is an organ selected from kidney, liver, spleen, and pancreas. In some embodiments, the tissues of origin comprise a tumor. In some embodiments, the cfDNA molecules are from one or more organs selected from skin, heart, kidney, liver, lungs, stomach, bladder or pancreas.

In some embodiments, the method further comprises treating the subject with an immunoregulatory agent when there is graft-versus-host disease in the subject.

In some embodiments, the biological sample is obtained from the subject at about 30 days post-HCT.

Another aspect of the disclosure is directed to a method for detecting microbial infection in a biological sample from a subject comprising: obtaining cell-free DNA (cfDNA) molecules from the biological sample; determining the sequences of the cfDNA molecules; and identifying the presence of a cfDNA sequence of a microbial species, thereby detecting an infection by the microbial species.

In some embodiments, prior to determining, a library of cfDNA molecules is prepared using single-stranded DNA (ssDNA) library preparation method.

In some embodiments, the cfDNA molecules are bisulfite treated before determining the sequences of the cfDNA molecules.

In some embodiments, the method further comprises treating the subject with an anti-microbial agent when a microbial cfDNA sequence is identified in the biological sample. In some embodiments, the anti-microbial agent is an anti-bacterial or anti-fungal agent. In some embodiments, the anti-microbial agent is an anti-viral agent.

In some embodiments, the subject has undergone hematopoietic cell transplantation (HCT).

In some embodiments, wherein the biological sample is a blood or a serum sample.

Another aspect of the disclosure is a method comprising: obtaining cfDNA molecules from a biological sample from a subject; determining the profiles of an epigenetic marker within the cfDNA molecules, wherein the epigenetic marker displays tissue-specific profiles; identifying the tissues of origin of the cfDNA molecules based on the profiles determined; measuring the level of cfDNA molecules from an identified tissue of origin, wherein an increased level of cfDNA molecules from said identified tissue of origin as compared to a control level is indicative of damage in said identified tissue of origin; and identifying the presence of a microbial cfDNA in the biological sample.

In some embodiments, the biological sample has been bisulfite treated.

In some embodiments, the identifying the presence of a microbial cfDNA in the biological sample comprises determining the sequences of the cfDNA molecules.

In some embodiments, the subject has undergone hematopoietic cell transplantation (HCT).

Another aspect of the disclosure is directed to method for detecting a tumor in a subject comprising: obtaining cell-free DNA (cfDNA) molecules from a biological sample from the subject; identifying the presence of a tumor-derived cfDNA molecule based on a tumor-specific DNA alteration; and measuring the level of the tumor-derived cfDNA molecule, wherein an increased level of tumor-derived cfDNA molecule as compared to a control level is indicative of the presence of tumor in the subject, or wherein an increased level of tumor-derived cfDNA molecule as compared to a level at an earlier time is indicative of tumor progression in the subject.

In some embodiments, prior to determining, a library of cfDNA molecules is prepared using single-stranded DNA (ssDNA) library preparation method.

In some embodiments, the tumor-specific DNA alteration is selected from a tumor-specific deletion, a tumor-specific amplification or a tumor-specific point mutation.

In some embodiments, the cfDNA molecules are bisulfite treated.

In some embodiments, the tumor-specific DNA alteration is tumor-specific DNA methylation.

In some embodiments, when an increased level of tumor-derived cfDNA molecules is detected, the method further comprises treating the subject with chemotherapy, a radiotherapy, or a combination therapy.

In some embodiments, the chemotherapy is selected from a DNA alkylating agent, an antimetabolite, an anti-tumor antibiotic, a topoisomerase inhibitor, a mitotic inhibitor, or a corticosteroid.

In some embodiments, the subject has undergone hematopoietic cell transplantation (HCT).

In some embodiments, the biological sample is a blood or a serum sample.

Another aspect of the disclosure is directed to a method for monitoring engraftment a subject who has undergone hematopoietic cell transplantation (HCT) from a donor comprising: obtaining cfDNA molecules from a biological sample from the subject; determining the profiles of a marker within the cfDNA molecules, wherein the marker has different profiles between the subject and the donor; identifying the origin of the cfDNA molecules based on the profiles determined; and measuring the level of cfDNA molecules from the subject and the level of cfDNA molecules from the donor, wherein an increased ratio of cfDNA molecules from the subject versus cfDNA molecules from the donor as compared to a control ratio is indicative of loss of engraftment.

In some embodiments, prior to determining, a library of cfDNA molecules is prepared using single-stranded DNA (ssDNA) library preparation method.

In some embodiments, the marker is selected from the group consisting of a sex chromosome, a DNA modification, a histone modification, and nucleosome positioning.

In some embodiments, the DNA modification is DNA methylation or DNA hydroxymethylation.

In some embodiments, the histone modification is selected from the group consisting of acetylation, methylation, phosphorylation, ubiquitylation, GlcNAcylation, citrullination, krotonilation, and isomerization.

In some embodiments, the determining the profiles of the epigenetic marker comprises determining the sequences of the cfDNA molecules. In some embodiments, the profile of DNA methylation is determined by bisulfite treatment or enzymatic DNA methylation analysis. In some embodiments, the profile of DNA hydroxymethylation is determined by a pull-down assay, a selective labeling assay, or an oxidative bisulfite sequencing assay.

In some embodiments, the profile of histone modification is detected by a pull-down assay. In some embodiments, the nucleosome positioning is determined by a nucleosome positioning assay.

In some embodiments, the determining the profiles of the epigenetic marker is achieved without determining the sequences of the cfDNA molecules.

In some embodiments, the determining is achieved by a PCR assay selected from quantitative PCR (qPCR) and digital droplet PCR (ddPCR). In some embodiments, the assay comprises amplifying cfDNA molecules from regions of the genome that have specific epigenetic markers.

In some embodiments, the biological sample is a blood, a plasma or a serum sample.

In some embodiments, the biological sample is obtained from the subject about 15 days, about 30 days, about 45 days, about 60 days, about 75 days, about 90 days, about 105 days, or about 120 days after the HCT.

In some embodiments, the control level is (i) the level of cfDNA molecules in a sample from the subject prior to HCT, or (ii) the level of cfDNA in a sample from a subject who has undergone HCT but who has not had loss of engraftment.

In some embodiments, the method further comprises treating the subject with an immunoregulatory agent when there is loss of engraftment.

In some embodiments, the biological sample is obtained from the subject at about 30 days post-HCT.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1F. Study workflow. (A) Blood samples from hematopoietic cell transplant recipients (n=18) were prospectively collected at six time points. (B) WGBS is performed on cfDNA extracted from patient plasma. Sequenced cfDNA is processed through a custom bioinformatics pipeline. (C), (D) hg19 sequence coverage (C) and bisulfite conversion efficiency (D) of sequenced cfDNA (n=106). Red lines indicate the median. (E) Fragment length profiles of 106 cfDNA samples after bisulfite treatment. Inset: Fourier analysis reveals a 10.4 bp periodicity in the fragment length profiles of bisulfite treated cfDNA. (F) UMAP dimensional reduction of cell and tissue methylation profiles. Individual tissues are colored by UMAP coordinates using a linear gradient where each of the four corners is either cyan, magenta, yellow or black.

FIGS. 2A-2C. Host-derived cfDNA dynamics before and after HCT. (A), (B) Effect of conditioning and HCT infusion on cfDNA composition (A) and absolute concentration (B). (C) Solid organ derived cfDNA concentration in plasma. Top row: dark lines represent mean solid-organ cfDNA and days post-transplant for each patient time point. Error Bars represent standard error of the mean. Bottom row: solid organ cfDNA by time point. Samples are removed from analysis if plasma was collected after aGVHD diagnosis. *p-value<0.05; **p-value<0.01.

FIGS. 3A-3D. Solid organ cfDNA concentration dynamics. (A) Solid organ cfDNA concentration in GVHD negative individuals. (B), (C), (D) Solid organ cfDNA concentration in three GVHD patients. Blue line represents loess-smoothed solid organ cfDNA in GVHD negative patients.

FIGS. 4A-4C. Plasma infectome. (A) Microbial cfDNA concentration by time point. (B) Polyomavirus, annelovirus and human herpesvirus abundance in plasma before and after HCT. (C) Human herpesvirus and polyomavirus species detected per patient (n=15) per time point (patients without detectable herpesvirus or human polyomavirus are not shown (n=3)). Error bars represent standard error of the mean.

FIGS. 5A-5G. (A) Schematic of potential sources of cell-free DNA in blood: Hematopoietic cell transplant (HCT) donor (blue), HCT recipient (i.e. the patient) non tumor tissue (orange), from tumor tissue (green, in the case of malignant disease). (B)-(C) Donor fraction measurements for patients over time. (B) Donor fraction measurements from sex-mismatched patients. Donor fractions are 0 before the transplant (as there is no donor-derived DNA, but is quite elevated at Engraftment (when there are clinical signs that the graft is producing a certain amount of blood cells)). (C) Two examples where patients experienced relapse (recurrence of blood disorder) that could be picked up via the donor fraction measurements. (D) Copy number alterations can be used to estimate a tumor fraction in cell-free DNA. Not all patients had malignant blood disorders with measurable copy-number alterations. However, when a tumor has copy number alterations, those alterations can be used to monitor the presence and progression of that tumor. Blue: malignant blood disorders; Orange: nonmalignant blood disorders. Lower limit of detection is determined empirically. (E)-(F) Genome-wide coverage plots made by mapping the detected cfDNA fragments to the human genome. (E) Patient 008 is an example of nonmalignant blood disorder and illustrates an example without any copy number alterations. (F) Patient 031 (an HCT recipient patient) is shown at three different time points: Pre-conditioning: prior to receiving HCT, Engraftment, and 6 months after engraftment. (G) Another example of a patient who had no copy number alterations at baseline, but suddenly lost one copy of chromosome 7.

### DETAILED DESCRIPTION

The inventors have developed novel methods for detecting tissue damage, graft-versus-host disease (GVHD), microbial infections, presence of a tumor, and loss of engraftment in a subject using cell-free DNA (cfDNA) profiling. The methods of the disclosure are in part based on the recognition that damaged tissues, microbes during an infection, tumors, and donor cells (e.g., in a hematopoietic cell transplantation) shed small fragments of cfDNA into blood circulation. The inventors found that the amount of cfDNA in the blood from a damaged tissue increases with increased damage to the tissue. The inventors found that during an infection microbial cfDNA circulates in the blood. In addition, the present disclosure is directed to methods for monitoring patients after hematopoietic cell transplantation (HCT) and detecting GVHD or loss of engraftment by cfDNA profiling.

### DEFINITIONS

### Biological Sample

The term “biological sample” includes body samples from an animal, including biological fluids such as serum, plasma, vitreous fluid, lymph fluid, synovial fluid, follicular fluid, seminal fluid, amniotic fluid, milk, whole blood, urine, cerebro-spinal fluid, saliva, sputum, tears, perspiration, mucus, and tissue culture medium, as well as tissue extracts such as homogenized tissue, and cellular extracts. In some embodiments, the biological sample is a serum, plasma or urine sample. The term “animal” includes mammals, for example, human, horse, camel, dog, cat, pig, cow, goat and sheep.

### Epigenetic Marker Profiling

As used herein, the term “epigenetic marker” refers to a characteristic of a nucleic acid or polypeptide that is not directly controlled by the genetic code. In some embodiments, the epigenetic marker is selected from the group consisting of a DNA modification, a histone modification, and nucleosome positioning.

In some embodiments, a DNA modification is DNA methylation or DNA hydroxymethylation.

In some embodiments, the determining the profiles of an epigenetic marker comprises determining the sequences of cfDNA molecules. In some embodiments, the profile of DNA methylation is determined by bisulfite treatment or enzymatic DNA methylation analysis.

In some embodiments, probing nucleosome positioning is achieved by sequencing cell-free DNA (without bisulfite treatment).

In some embodiments, the profile of DNA hydroxymethylation is determined by a pull-down assay, a selective labeling assay, or an oxidative bisulfite sequencing assay.

In some embodiments, the profile of histone modification is detected by a pull-down assay.

In some embodiments, the histone modification is selected from the group consisting of acetylation, methylation, phosphorylation, ubiquitylation, GlcNAcylation, citrullination, krotonilation, and isomerization.

In some embodiments, the nucleosome positioning is determined by a nucleosome positioning assay.

In some embodiments, the determining the profiles of the epigenetic marker is achieved without determining the sequences of the cfDNA molecules.

As used herein, the phrase “bisulfite treatment” refers to a reaction for the conversion of a non-methylated cytosines in a nucleic acid to uracil bases in the presence of bisulfite ions. 5-methyl-cytosine bases are not significantly converted to uracil bases during bisulfite treatment. Grunau, C., et al. (Nucleic Acids Res, 29 (2001) e65-5, pp 1-7), which is incorporated herein in its entirety, discloses experimental parameters of bisulfite treatment.

Enzymatic analysis of DNA methylation relies on restriction enzymes that are methylation sensitive. In some embodiments, the methylation sensitive enzyme is HpaI, which recognizes and cuts GTT\hat{ }AAC sites when unmethylated. In some embodiments, the methylation sensitive enzyme is HpaII. HpaII does not cut its CCGG recognition site if it is methylated. MspI will cut CCGG recognition site regardless of methylation. When examining a region with a CCGG site, if the fragment is cut by HpaII then it was unmethylated, if uncut then it was methylated. The sample is also digested with MspI as a control for proper digestion. In some embodiments, several closely related enzymatic analysis techniques use these enzymes. In the HpaII tiny fragment Enrichment by Ligation-mediated PCR (HELP) assay, adapters are ligated to the HpaII and MspI digested DNA followed by and PCR amplification. The fragments are then identified using microarray (HELP-chip) or sequencing (HELP-seq). In some embodiments, the enzymatic analysis technique is RLGC (Restriction Landmark Genomic Scanning) which involves running the fragments on a two-dimensional gel to detect methylation of many regions simultaneously. In some embodiments, the enzymatic analysis technique is DNA methylation Restriction Enzyme Analysis (MSRE), which is very similar to HELP but uses real-time PCR. Details of several enzymatic analysis techniques are found in Suzuki, M., and Greally, J. M. ((2010). DNA methylation profiling using tiny fragment enrichment by ligation-mediated PCR (HELP). Methods 52, 218-222); Oda, M., and Greally, J. M. ((2009). The HELP assay. Methods Mol. Biol. 507, 77-87); Koike, K. et al., ((2008). Epigenetics: application of virtual image restriction landmark genomic scanning (Vi-RLGS). FEBS J. 275, 1608-1616); and Ando, Y., and Hayashizaki, Y. ((2006). Restriction landmark genomic scanning Nat. Protoc. 1, 2774-2783), which are incorporated herein in their entireties.

In some embodiments, the epigenetic profiling comprises alternatives to methylation markers. In some embodiments, other epigenetic marks that are tissue specific and maintained in cell-free DNA are used in the methods of this disclosure.

In some embodiments, the epigenetic profiling comprises DNA hydroxymethylation profiling. Hydroxymethylation is a chemical modification present on cytosines that is thought to be indicative of a gene being activated. Papers have described this chemical modification as tissue-specific. See, e.g., Song, Chun-Xiao, et al., Cell Research, 27.10 (2017): 1231-1242; Nestor, Colm E., et al., Genome research; 22.3 (2012): 467-477, incorporated herein in their entirety.

In some embodiments, determining hydroxymethylation profile of cfDNA is achieved by a pull-down assay. In some embodiments, the pull-down assay utilizes engineered antibodies specific for hydroxymethylated cytosines can be used to capture hydroxymethylated-rich cell-free DNA.

In some embodiments, determining hydroxymethylation profile of cfDNA is achieved by a selective labeling assay (selective chemical labelling of hydroxymethylated cytosines). In some embodiments, selective labeling is achieved by a B-glucosyltransferase enzyme that adds a biotin group to the hydroxymethylated cytosines. Streptavidin beads can then be ligated to the biotin groups to pull out hydroxymethylated-rich cfDNA. The pulled down cfDNA is then sequenced

In some embodiments, determining hydroxymethylation profile of cfDNA is achieved by oxidative bisulfite sequencing. In some embodiments, oxidative bisulfite sequencing comprises a) splitting cfDNA into two groups; b) bisulfite-treating one half of the cfDNA sample, revealing which cytosines were methylated or hydroxymethylated; c) oxidizing the other half (which removes the hydroxymethyl group), and then bisulfite treating the oxidized half. This reveals which cytosines were methylated; and d) Sequencing the split samples reveals which cytosines were methylated, and which were hydroxymethylated. Gives a single-base pair resolution to both hydroxymethylated and methylated sites

In some embodiments, the epigenetic profiling comprises histone modifications. DNA in the genome is wrapped around nucleosomes. Nucleosomes are composed of 8 histones. Histone modifications are associated with gene expression and their presence can be detected to estimate the tissues of origin of cell free DNA. When DNA is out of the cell (through apoptosis, for example), it gets degraded. It is thought that DNA that is wrapped around histones, however, is more protected from degradation, and can therefore be captured and sequenced (most cfDNA we sequenced is histone-wrapped). The modifications on these histones are indicative of tissues of origin. See, Sadeh, Ronen, et al., bioRxiv (2019): 638643, incorporated herein in its entirety.

In some embodiments, probing histone modifications in cell free DNA is achieved by:

1—Performing a pull-down assay using antibodies specific for a histone modification. In some embodiments, the antibodies are specific for histone methylation, acetylation, phosphorylation, ubiquitylation, GlcNAcylation, citrullination, krotonilation, or isomerization. In some embodiments, the histone methylation-specific antibodies comprise antibodies against H3K4Me1, H3K4Me2, H3K4Me3, or H3K36Me3 modifications.

2—Sequencing pulled-down cfDNA.

In some embodiments, probing histone modifications in cell free DNA is achieved by nucleosome positioning. DNA that is wrapped in a nucleosome cannot be transformed into RNA. It needs to be unwrapped by enzymes to be transcribed. Therefore, when a cell dies and its genome is released, the areas that were being transcribed are degraded (because they are not transcribed). The cfDNA we do sequence was not being transcribed. In theory, the areas of the genome that were not seen can be assumed to be actively transcribed. These patterns have been shown to be tissue-specific. See, Snyder, Matthew W., et al., Cell, 164.1-2 (2016): 57-68.; Sun, Kun, et al., Genome Research, 29.3 (2019): 418-427., incorporated in their entirety.

### Determining Epigenetic Profiles without Sequencing

In some embodiments, epigenetic profiling can be determined without sequencing the cfDNA. In some embodiments, epigenetic profiling is determined by performing quantitiative PCR (qPCR) or digital droplet PCR (ddPCR) (as described in Shemer, R. et al. Current Protocols in Molecular Biology, 127.1 (2019): e90; and Zemmour, Hai, et al., Nature Communications, 9.1 (2018): 1-9, both incorporated herein in their entirety). In some embodiments, epigenetic profiling involves isolating cell-free DNA from a sample; amplifying cfDNA from regions of the genome that have specific epigenetic markers; detecting modified or unmodified epigenetic marks at a tissue-specific region using probes that can distinguish modified and unmodified epigenetic marks; and using a either a qPCR or ddPCR assay to detect a readout. In some embodiments, the primers and/or probes comprise fluorescent labels. In some embodiments, the fluorescent signal from the probe is measured as the readout and tissue composition of the cfDNA is inferred from the readout.

In some embodiments, the epigenetic profile is a DNA methylation profile. In some embodiments, determining the methylation profile does not comprise determining the sequence of the cfDNA.

In some embodiments, after bisulfite treatment, qPCR or ddPCR are used to amplify regions that comprise specific methylation markers that are tissue specific. The degree of amplification can be measured to estimate tissue-specific contributions to cell-free DNA.

In some embodiments, determining methylation profiles comprise isolating cell-free DNA from a sample; amplifying cfDNA from regions of the genome that have methylation-specific markers; detecting methylation at a tissue-specific region; and using a either a qPCR or ddPCR assay to readout the fluorescent signal, and use the fluorescent signal to infer tissue composition of cfDNA. In some embodiments, determining methylation at a tissue-specific region is achieved by using probes that bind to either methylated or unmethylated cytosines.

### Immunoregulatory Agent

As used herein, the phrase “immunoregulatory agent” refers to an agent that regulates the activity of the immune system. In some embodiments, an immunoregulatory agent suppresses immune responses.

In some embodiments, the immunoregulatory agent is selected from an anti-inflammatory drug, a steroid (e.g., glucocorticoid), an antibody or a small molecule drug.

In some embodiments, the steroid drug is selected from hydrocortisone, cortisone, ethamethasoneb, prednisone, prednisolone, triamcinolone, methylprednisolone, or dexamethasone.

In some embodiments, the antibody drug is selected from interleukin-2 receptor antibodies, brentuximab, alemtuzumab, or tocilizumab.

In some embodiments, the small molecule drug is selected from tacrolimus, sirolimus, ciclosporin, zotarolimus, or everolimus. The term “small molecule” herein refers to small organic chemical compound, generally having a molecular weight of less than 2000 daltons, less than 1500 daltons, less than 1000 daltons, less than 800 daltons, or less than 600 daltons.

In some embodiments, the immunoregulatory agent is selected from ruxolitinib, ibrutinib, mycophenolate mofetil, etanercept, pentostatin, alpha-1 antitrypsin, sirolimus, extracorporeal photopheresis, anti-thymocyte globulin, mesenchymal stromal cells and monoclonal antibodies such as interleukin-2 receptor antibodies, brentuximab, alemtuzumab, or tocilizumab.

### Methods for Detecting Tissue Damage

The inventors have recognized that when there is tissue damage, cfDNA molecules are released from the damaged tissue(s). The inventors also recognized that the amount of tissue-specific cfDNA is correlated to the amount of tissue damage, i.e., the more damaged a tissue is, the more cfDNA it releases. Each tissue type has a specific and distinct epigenetic marker profile that is different than other tissue types, and one can determine the source of a cfDNA using the epigenetic marker profile of the cfDNA.

An aspect of this disclosure is directed to a method for detecting tissue damage in a subject comprising obtaining cfDNA molecules from a biological sample from the subject; determining the profiles of an epigenetic marker within the cfDNA molecules, wherein the epigenetic marker displays tissue-specific profiles; identifying the tissues of origin of the cfDNA molecules based on the profiles determined; and measuring the level of cfDNA molecules from an identified tissue of origin, wherein (i) the level or (ii) an increased level of cfDNA molecules from said identified tissue of origin as compared to a control level, is indicative of damage in said identified tissue of origin.

In some embodiments, prior to determining, a library of cfDNA molecules is prepared using single-stranded DNA (ssDNA) library preparation method.

In some embodiments, the determining is achieved by a PCR assay selected from quantitative PCR (qPCR) and digital droplet PCR (ddPCR).

In some embodiments, the assay comprises amplifying cfDNA molecules from regions of the genome that have specific epigenetic markers.

In some embodiments, the subject has undergone hematopoietic cell transplantation (HCT).

In some embodiments, the biological sample is a blood or a serum sample.

In some embodiments, the biological sample is obtained from the subject about 15 days, about 30 days, about 45 days, about 60 days or about 90 days after the HCT.

In some embodiments, the control level is (i) the level of cfDNA molecules in a sample from the subject prior to HCT, or (ii) the level of cfDNA molecules in a sample from a subject who has undergone HCT but who has not had graft-versus-host disease (GVHD).

In some embodiments, the tissues of origin comprise a solid organ. In some embodiments, the solid organ is an organ selected from kidney, liver, spleen, or pancreas. In some embodiments the tissues of origin comprise a tissue from one or more of lung, stomach, small intestine, large intestine, skin, heart, kidney, liver, bladder and pancreas. In some embodiments, the tissues of origin comprise a tumor tissue.

In some embodiments the cfDNA molecules are from one or more organs selected from lung, stomach, small intestine, large intestine, skin, heart, kidney, liver, bladder or pancreas.

In some embodiments, when there is a tissue damage in the subject, the method further comprises treating the subject with a therapy to ameliorate a damaged tissue in the subject. In some embodiments, the therapy comprises administering to the subject an immunoregulatory agent.

In some embodiments, detecting tissue damage at about 30 days post-HCT is indicative of organ rejection or a risk of developing organ rejection. In some embodiments, detecting tissue damage is indicative of graft-versus-host disease (GVHD). In some embodiments, when there is GVHD in the subject, the method further comprises treating the subject with an immunoregulatory agent.

In some embodiments, detecting tissue damage is indicative of a microbial infection. In some embodiments, when microbial infection is implicated, the method further comprises administering to the subject an antibiotic or antiviral drug suitable to treat the microbial infection.

In some embodiments, detecting tissue damage is indicative of drug toxicity. In some embodiments, when drug toxicity is implicated, the drug suspected of toxicity is discontinued or its dose is reduced.

### Methods for Monitoring HCT Patients for GVHD

A potential problem for HCT recipients (patients) is GVHD, where donor immune cells attack and damage host tissues and organs. It is very important to monitor HCT patients to detect GVHD before serious or permanent damage occurs.

Another aspect of the disclosure is directed to a method for monitoring a subject who has undergone HCT, comprising obtaining cfDNA molecules from a biological sample from the subject; determining the profiles of an epigenetic marker within the cfDNA molecules, wherein the epigenetic marker displays tissue-specific profiles; identifying the tissues of origin of the cfDNA molecules based on the profiles determined; and measuring the level of cfDNA molecules from an identified tissue of origin, wherein an increased level of cfDNA molecules from said identified tissue of origin as compared to a control level is indicative of graft-versus-host disease.

In some embodiments, prior to determining, a library of cfDNA molecules is prepared using single-stranded DNA (ssDNA) library preparation method.

In some embodiments, the determining is achieved by a PCR assay selected from quantitative PCR (qPCR) and digital droplet PCR (ddPCR).

In some embodiments, the assay comprises amplifying cfDNA molecules from regions of the genome that have specific epigenetic markers.

In some embodiments, the biological sample is a blood or a serum sample.

In some embodiments, the biological sample is obtained from the subject about 15 days, about 30 days, about 45 days, about 60 days or about 90 days after the HCT.

In some embodiments, the control level is (i) the level of cfDNA molecules in a sample from the subject prior to HCT, or (ii) the level of cfDNA molecules in a sample from a subject who has undergone HCT but who has not had graft-versus-host disease (GVHD).

In some embodiments, the tissues of origin comprise a solid organ. In some embodiments, the solid organ is an organ selected from kidney, liver, spleen, or pancreas. In some embodiments the tissues of origin comprise a tissue from one or more of lung, stomach, small intestine, large intestine, skin, heart, kidney, liver, bladder and pancreas. In some embodiments, the tissues of origin comprise a tumor tissue.

In some embodiments the cfDNA molecules are from one or more organs selected from lung, stomach, small intestine, large intestine, skin, heart, kidney, liver, bladder or pancreas.

In some embodiments, detecting tissue damage at about 30 days post-HCT is indicative of organ rejection or a risk of developing organ rejection. In some embodiments, detecting tissue damage is indicative of graft-versus-host disease (GVHD). In some embodiments, when there is GVHD in the subject, the method further comprises treating the subject with an immunoregulatory agent.

### Methods for Monitoring HCT Patients for Loss of Engraftment

Another potential issue for HCT patients is loss of the donor hematopoietic cells, also known as loss of engraftment, which may or may not be followed by the relapse of the blood cancer.

Another aspect of the disclosure is directed to a method for monitoring engraftment a subject who has undergone hematopoietic cell transplantation (HCT) from a donor comprising: obtaining cfDNA molecules from a biological sample from the subject; determining the profiles of a marker within the cfDNA molecules, wherein the marker has different profiles between the subject and the donor; identifying the origin of the cfDNA molecules based on the profiles determined; and measuring the level of cfDNA molecules from the subject and the level of cfDNA molecules from the donor, wherein an increased ratio of cfDNA molecules from the subject versus cfDNA molecules from the donor as compared to a control ratio is indicative of loss of engraftment.

In some embodiments, prior to determining, a library of cfDNA molecules is prepared using single-stranded DNA (ssDNA) library preparation method.

In some embodiments, the determining is achieved by a PCR assay selected from quantitative PCR (qPCR) and digital droplet PCR (ddPCR).

In some embodiments, the assay comprises amplifying cfDNA molecules from regions of the genome that have specific epigenetic markers.

In some embodiments, the biological sample is a blood or a serum sample.

In some embodiments, the biological sample is obtained from the subject about 15 days, about 30 days, about 45 days, about 60 days or about 90 days after the HCT.

In some embodiments, the control level is (i) the level of cfDNA molecules in a sample from the subject prior to HCT, or (ii) the level of cfDNA molecules in a sample from a subject who has undergone HCT but who has not had loss of engraftment.

In some embodiments, the method further comprises treating the subject with an immunoregulatory drug when there is loss of engraftment.

### Methods for Detecting Microbial Infections

Another aspect of this disclosure is directed to a method for detecting microbial infection in a biological sample from a subject comprising obtaining cell-free DNA (cfDNA) molecules from the biological sample; determining the sequences of the cfDNA molecules; and identifying the presence of a cfDNA sequence of a microbial species, thereby detecting an infection by the microbial species.

In some embodiments, prior to determining, a library of cfDNA molecules is prepared using single-stranded DNA (ssDNA) library preparation method.

In some embodiments, the method further comprises treating the subject with an anti-microbial agent when a microbial cfDNA sequence is identified in the biological sample.

In some embodiments, the anti-microbial agent is an anti-bacterial or anti-fungal agent. In some embodiments, the anti-microbial agent is an anti-viral agent.

In some embodiments, the subject has undergone hematopoietic cell transplantation (HCT).

In some embodiments, the biological sample is a blood or a serum sample

Another aspect of the disclosure utilizes epigenetic marker profile analysis to detect microbial infections.

In some embodiments, the disclosure is directed to a method comprising obtaining cfDNA molecules from a biological sample from a subject; determining the profiles of an epigenetic marker within the cfDNA molecules, wherein the epigenetic marker displays tissue-specific profiles; identifying the tissues of origin of the cfDNA molecules based on the profiles determined; measuring the level of cfDNA molecules from an identified tissue of origin, wherein an increased level of cfDNA molecules from said identified tissue of origin as compared to a control level is indicative of damage in said identified tissue of origin; and identifying the presence of a microbial cfDNA in the biological sample.

In some embodiments, the identifying the presence of a microbial cfDNA in the biological sample comprises determining the sequences of the cfDNA molecules. In some embodiments, the subject has undergone hematopoietic cell transplantation (HCT).

### Methods for Detecting or Monitoring a Tumor in a Subject

Another aspect of this disclosure is directed to a method for detecting a tumor in a subject comprising obtaining cell-free DNA (cfDNA) molecules from a biological sample from the subject; identifying the presence of a tumor-derived cfDNA molecule based on a tumor-specific DNA alteration; and measuring the level of the tumor-derived cfDNA molecule, wherein an increased level of tumor-derived cfDNA molecule as compared to a control level is indicative of the presence of tumor in the subject, or wherein an increased level of tumor-derived cfDNA molecule as compared to a level at an earlier time is indicative of tumor progression in the subject.

In some embodiments, prior to determining, a library of cfDNA molecules is prepared using single-stranded DNA (ssDNA) library preparation method.

In some embodiments, the tumor-specific DNA alteration is selected from a tumor-specific deletion, a tumor-specific amplification or a tumor-specific point mutation.

In some embodiments, the cfDNA molecules are bisulfite treated.

In some embodiments, the tumor-specific DNA alteration is tumor-specific DNA methylation.

In some embodiments, when an increased level of tumor-derived cfDNA molecules is detected, the method further comprises treating the subject with chemotherapy, a radiotherapy, or a combination therapy.

In some embodiment, the chemotherapy alkylating agent (e.g., nitrosoureas), an antimetabolite, an anti-tumor antibiotic (e.g., anthracyclines), a topoisomerase inhibitor, a mitotic inhibitor (e.g., taxanes and vinca alkaloids), or a corticosteroid.

In some embodiment, the subject has undergone hematopoietic cell transplantation (HCT).

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The specific examples listed below are only illustrative and by no means limiting.

### EXAMPLES

### Example 1: Materials and Methods

### Study Cohort

A nested case-control study was performed within a prospective cohort of adult patients undergoing allogeneic HCT at Dana-Farber Cancer Institute. Patients were followed for 6 months after HCT. Patients were selected for this study on a rolling basis, and were placed in the GVHD case or control groups based on clinical manifestation of the disease within the first 6 months after HCT. Individuals were excluded from the study if they did not provide blood samples for at least 5 of the 6 studied time points (pre-conditioning, day of transplant, engraftment, months 1, 2 and 3). The study was approved by the Dana-Farber/Harvard Cancer Center's Office of Human Research Studies. All patients provided written informed consent.

For this study, 106 blood samples collected from 18 allogeneic HCT recipients from August 2018- to April -2019 were used. Baseline patient characteristics were recorded. Covariates of interest included HLA matching, donor relatedness and donor-recipient sex mismatch. Date of onset of GVHD, as well as GVHD prophylaxis and treatment regimens were documented. GVHD was diagnosed clinically and pathologically. GVHD severity was graded according to the Glucksberg criteria. Other clinical events of interest included the development of bloodstream infections, BK polyomavirus disease, and clinical disease from other DNA viruses.

### Engraftment

Neutrophil engraftment was considered when blood samples contained an absolute neutrophil count greater or equal than 500 cell per microliter of blood on two separate measurements.

### BK Polyomavirus Disease Identification

Patients were identified as BK virus disease positive when they presented BK-related urinary symptoms that correlated with positive BK qPCR test in either urine or blood (>10⁵ copies/mL in urine, >0 copies/mL in blood; Viracor BK qPCR test, reference #2500) and did not have evidence of any other cause of genitourinary pathology at the time of symptom onset.

### Blood Sample Collection and Plasma Extraction

Blood samples were collected through standard venipuncture in EDTA tubes (Becton Dickinson (BD), reference #366643) on admission, before the beginning of the conditioning chemotherapy; on the day of HCT after the completion of the conditioning chemotherapy, at engraftment (usually 14 to 21 days after HCT), and at months 1, 2, and 3 post-HCT. Plasma was extracted through blood centrifugation (2000 rpm for 10 minutes using a Beckman Coulter Allegra 6R centrifuge) and stored in 0.5-2 mL aliquots at −80° C. Plasma samples were shipped from DFCI to Cornell University on dry ice.

### Nucleic Acid Control Preparation

Synthetic oligos were prepared (IDT), mixed in equal proportions, and diluted at approximately 150 ng/ul. At the time of cfDNA extraction, 8 μ1 of control was added to 1992 μL of 1xPBS and processed as a sample in all downstream experiments.

### Cell-Free DNA Extraction

cfDNA was extracted according to manufacturer recommendations (Qiagen Circulating Nucleic Acid Kit, reference #55114, elution volume 45 μl). Eluted DNA was quantified using a Qubit 3.0 Fluorometer (using 2 μL of eluted DNA). Measured cfDNA concentration was obtained using the following formula:

cfDNA concentration=((Eluted cfDNA concentration)*(Elution volume))/((Plasma volume))

Whole-genome bisulfite sequencing. cfDNA and nucleic acid controls were bisulfite treated according to manufacturer recommendations (Zymo Methylation Lightning Kit, reference #D5030). Sequencing libraries were prepared using a previously described single-stranded library preparation protocol21. Libraries were quality-controlled through DNA fragment analysis (Agilent Fragment analyzer) and sequenced on an Illumina NextSeq550 machine using 2×75 bp reads. Nucleic acid controls were sequenced a ˜1% of the total sequencing lane.

### Human Genome Alignment

Adapter sequences were trimmed using BBTools. The Bismark alignment tool was used to align reads to the human genome (version hg19), remove PCR duplicates and calculate methylation densities.

Reference tissue methylation profiles and tissue of origin measurement. Reference tissue methylomes were obtained from publicly available. Genomic coordinates from different sources were normalized and converted to a standard 4 column bed file (columns chromosome, start, end, methylation fraction) using hg19 assembly coordinates. Methylation profiles were grouped by tissue-type and differentially methylated regions were found using Metilene. Tissues and cell-types of origin were determined using quadratic programming

### Metagenomic Alignment and Quantification of Microbial cfDNA

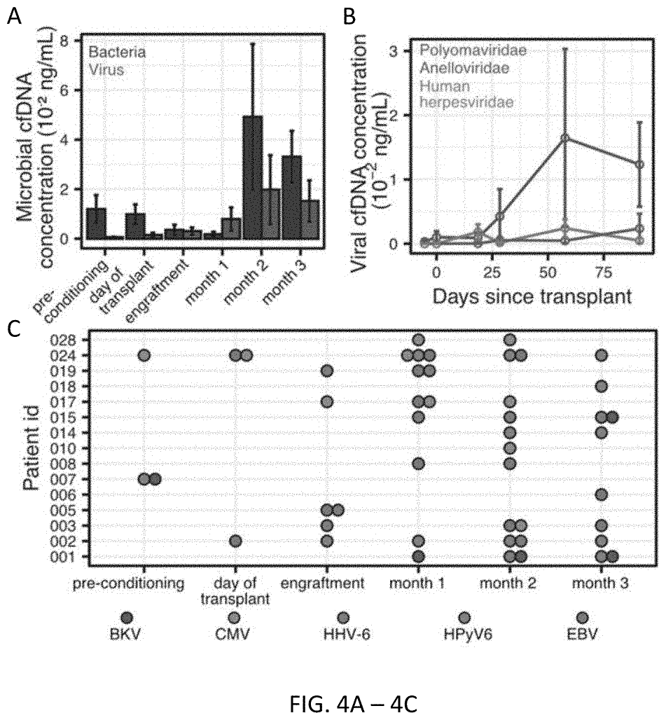

After WGBS, reads were adapter-trimmed using BBTools, and short reads are merged with FLASH. Sequences were aligned to a C-to-T converted genome using Bismark. Unmapped reads were BLASTed using hs-blastn to a list of C-to-T converted microbial reference genomes. A relative abundance of all detected organisms was determined using GRAMMy, and relative genomic abundances are measured. Microbial cfDNA fraction was calculated by dividing the unique number of reads mapping to microbial species (after adjusting for the length of each microbial genome in the reference set) to the total number of sequenced reads. Human fraction is estimated as 1—microbial fraction. Microbial species were then filtered for environmental contamination and alignment noise using previously described methods (LBBC, PNAS). For viral species identification (FIG. 4C), custom genomic reference was generated from representative genomes for BK polyomavirus (NC_001538.1), cytomegalovirus (NC_006273.1), herpesvirus 6A (NC_001664.4) and 6B (NC_00898.1), human polyomavirus 6 (NC_014406.1) and 7 (NC_014407.1) and Epstein-Barr virus (NC_007605). Reads that BLASTed to these species were then re-aligned via Bismark, and a threshold of 1 mapped sequence per 40 million total sequenced reads was used to positively identify a viral species within a plasma sample.

### cfDNA Concentration

cfDNA concentration of a specific tissue or microbe is calculated as follows:

Normalized cfDNA concentration=((cfDNA concentration)*(Nucleic acid control input mass))/(Nucleic acid control output mass)

Tissue specific cfDNA concentration=(Normalized cfDNA concentration)*(human read fraction)*(tissue proportion)

Microbial cfDNA concentration=(Normalized cfDNA concentration)*(microbial read fraction)

### Depth of Coverage

The depth of sequencing was measured by summing the depth of coverage for each mapped base pair on the human genome after duplicate removal, and dividing by the total length of the human genome (hg19, without unknown bases).

### Bisulfite Conversion Efficiency

Bisulfite conversion efficiency was estimated by quantifying the rate of C[A/T/C] methylation in human-aligned reads (using MethPipe), which are rarely methylated in mammalian genomes.

### Statistical Analysis

Statistical analysis was performed in R (version 3.5). All tests were performed using a two-sided Wilcoxon test.

### Example 2

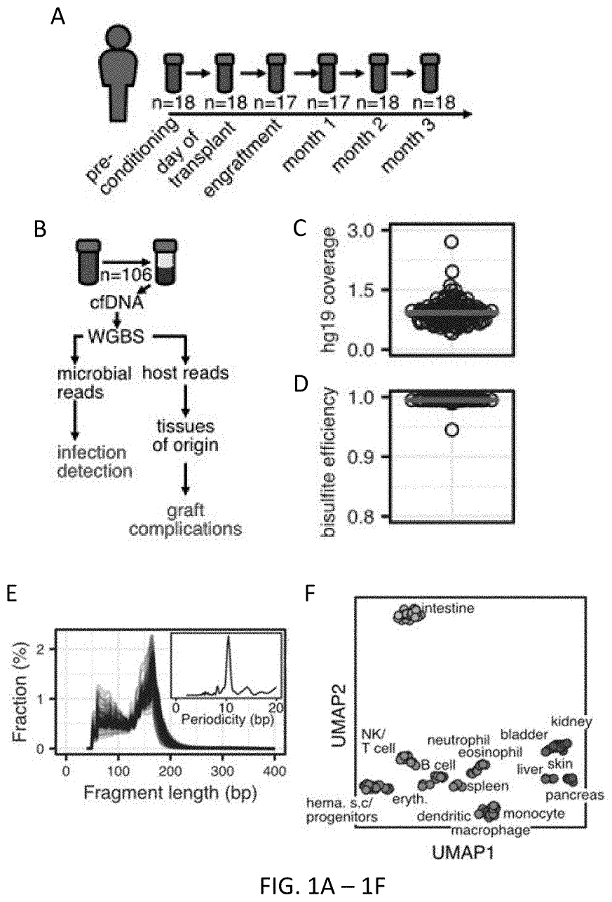

The inventors performed a prospective cohort study to evaluate the utility of cfDNA to predict and monitor complications after allogeneic HCT. For this study, the inventors selected 18 adults that underwent allogeneic HCT and assayed a total of 106 serial plasma samples collected at six predetermined time points, including before conditioning chemotherapy, on the day of but before hematopoietic cell infusion, after neutrophil engraftment (>500 neutrophils per microliter), and at one, two, and three months post HCT (FIG. 1A). The test cohort included patients with both malignant (n=14) and non-malignant blood disorders (n=4). In total, nine patients developed acute GVHD (GVHD+) and nine did not (GVHD−), two developed a bloodstream infection and two developed BK virus disease.

The inventors isolated cfDNA from plasma (0.5 mL-1.9 mL per sample) and implemented whole-genome bisulfite sequencing to profile cytosine methylation marks comprised within cfDNA (FIG. 1B). A single-stranded DNA (ssDNA) library preparation was used to obtain sequence information after bisulfite conversion. This ssDNA library preparation avoids degradation of adapter-bound molecules which is common for WGBS library preparations that rely on ligation of methylated adapters before bisulfite conversion and avoids amplification biases inherent to WGBS library preparations that implement random priming. The inventors obtained 41±15 million paired-end reads per sample, corresponding to 0.9±0.3 fold per-base human genome coverage (FIG. 1C) and achieved a high bisulfite conversion efficiency (99.4%±0.5%, FIG. 1D). Paired-end read mapping was used to characterize the length of bisulfite treated cfDNA at single-nucleotide resolution and to investigate potential degradation of cfDNA due to bisulfite treatment. This analysis revealed a fragmentation profile similar to the fragmentation profile for plasma cfDNA that was not subjected to bisulfite treatment. The mode of fragments longer than 100 bp was 165 bp±7 bp (FIG. 1E), and Fourier analysis revealed a 10.4 bp periodicity in the fragment length profile (FIG. 1E, inset). A second peak at 60-90 bp in the fragment length profile is characteristic of single-stranded library preparation methods and was reported previously. Overall, the inventors do not find evidence of significant cfDNA fragmentation due to bisulfite treatment.

### Temporal Dynamics in Response to Conditioning Therapy and HCT

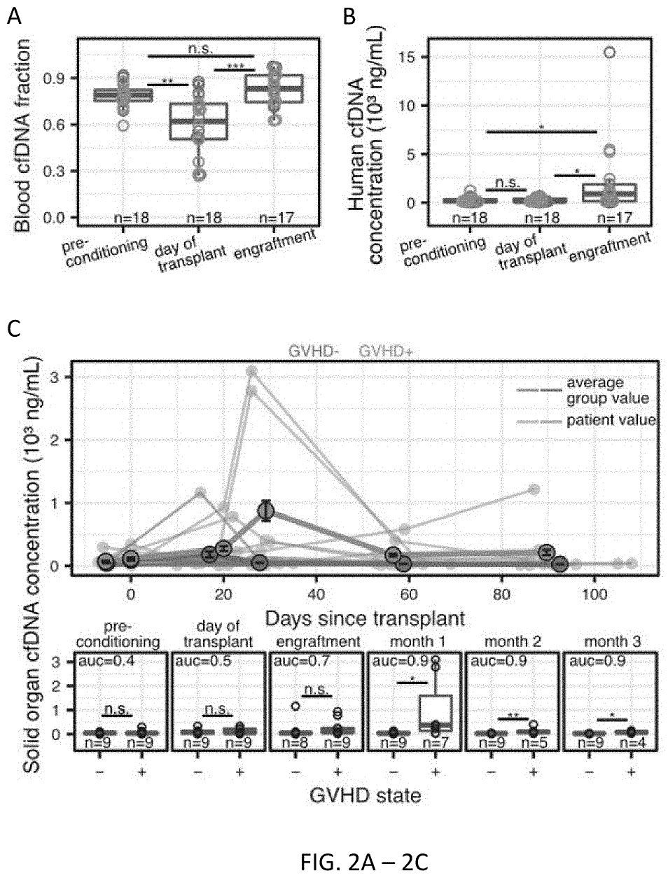

To quantify the relative proportion of cfDNA derived from different vascularized tissues and hematologic cell types, the inventors analyzed cfDNA methylation profiles against a reference set of methylation profiles of pure cell and tissue types (138 reference tissues, see Methods, and FIG. 1F). The inventors computed the absolute concentration of tissue-specific cfDNA by multiplying the proportion of tissue-specific cfDNA with the concentration of total host-derived cfDNA (see Methods in Example 1). FIGS. 2A-2C summarize these measurements for all patients and time points and reveal rich dynamics in tissue-origin of cfDNA in response to both conditioning chemotherapy and HCT (FIGS. 2A-2C). The most striking features seen in the data include i) a decrease in blood-cell specific cfDNA in response to conditioning therapy performed to deplete the patient's own immune cells, as expected (FIG. 1G, FIG. 2A), ii) an increase in total cfDNA concentration at engraftment (FIG. 1H, FIG. 2B), iii) a decrease in total cfDNA concentration after 60 days for most patients (FIG. 1H), and iv) an association between tissue-specific cfDNA and the incidence of GVHD.

The inventors next examined these features in more detail to explore the utility of these measurements to monitor immune related complications of HCT (FIGS. 2A-2C). Prior to conditioning, neutrophils, erythrocyte progenitors and monocytes were the major contributors of cfDNA in plasma (23.0%, 12.3% and 11.7%, respectively, average cfDNA concentration 272±305 ng/mL plasma). A variety of HCT conditioning regimens have been developed with varying degrees of organ toxicity and myelosuppression. The majority of patients in the cohort received reduced intensity conditioning therapy (RIC, n=17), whereas a single patient received myeloablative conditioning therapy (id number 005). Comparison of cfDNA tissues-of-origin in plasma before and after conditioning showed a significant drop in blood-derived cfDNA as expected from the function of the conditioning therapy (mean proportion of hematopoietic cell cfDNA decreased from 79%±10% to 59%±20%, p-value=0.0014, FIG. 2A). The proportion of blood-derived cfDNA increased to 84% ±10% at engraftment (p-value=5.6×10-5, FIG. 2A). The most notable effect of stem cell infusion and engraftment was a significant increase in the absolute concentration of cfDNA (mean human-derived cfDNA concentration from 256 ng/mL on day of transplant to 2149 ng/mL at engraftment [p-value=0.013], FIG. 2B).

### Performance Analysis

The inventors next evaluated the performance of a cfDNA tissue-of-origin measurement to predict GVHD (FIG. 2C). The inventors defined GVHD here as the clinical manifestation of any stage of the disease within the first 6 months post HCT (GVHD+, see Methods in Example 1). The inventors excluded samples collected after GVHD diagnosis, as these patients received additional GVHD treatment. The inventors found that the concentration of solid-organ specific cfDNA was significantly elevated for patients in the GVHD+ group at engraftment, month 1, 2 and 3 (p-values of 0.17, 0.012, 0.0070, 0.020, respectively), but not at the two pre-transplant time points (p=0.66 prior to conditioning, and p=0.80 prior to hematopoietic cell infusion). Receiver operating characteristic analysis of the performance of cfDNA as a predictive marker of GVHD yielded an area under the curve (AUC) of 0.7, 0.9, 0.9 and 0.9 at engraftment and months 1, 2, and 3, respectively. These results support the notion that cfDNA predicts GVHD occurrence as early as one month after HCT (mean solid organ cfDNA of 995 and 50 ng/mL plasma for GVHD+ and GVHD−, respectively; AUC=0.9, p-value<0.012, FIG. 2C).

To evaluate the ability of this assay to pinpoint the site of incidence of GVHD, the inventors quantified the burden of skin-derived cfDNA in the blood of GVHD negative individuals (n=9) and individuals who developed cutaneous GVHD (n=8). The inventors found that plasma samples from individuals with GVHD had a higher burden of skin-derived cfDNA when compared to samples from individuals who did not develop cutaneous GVHD (mean skin cfDNA of 20.6 ng/mL plasma and 3.2 ng/mL plasma, respectively, p-value=0.015 for samples collected post-transplant and pre-diagnosis). The number of samples from patients diagnosed with hepatic and gastrointestinal GVHD was insufficient to test the performance of the assay to pinpoint GVHD related injury to the liver or gut (n=1 and n=3, respectively).

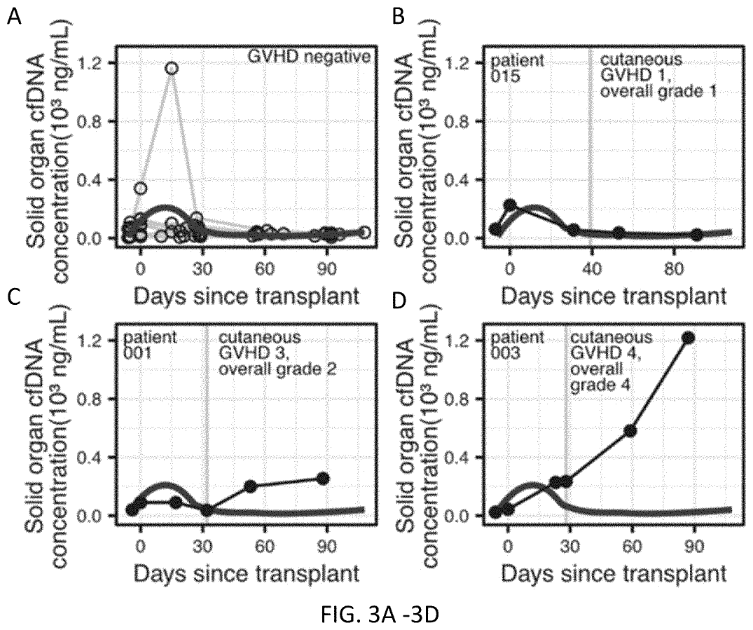

The inventors next studied the response to GVHD treatment for three similar patients for which samples and cfDNA tissues-of-origin analyses were available after GVHD diagnosis (male patients with RIC chemotherapy and similar GVHD diagnosis timepoints). These patients were diagnosed with GVHD between days 28 and 39 post HCT and two plasma samples after diagnosis were available for each patient. The first patient was diagnosed with mild GVHD (cutaneous stage 1, overall grade I; resolved day 98), and the tissue-of-origins of cfDNA followed a similar pattern observed for GVHD negative patients (FIGS. 3A-3B). The second patient was diagnosed with moderate GVHD (cutaneous stage 3, overall grade II; resolved day 137). cfDNA tissue-of-origin profiling identified an increase in solid-organ derived cfDNA after diagnosis (36.5 ng/mL at diagnosis, 199.4 ng/mL and 254.1 ng/mL at months 2 and 3, respectively; FIG. 3C). The third patient was diagnosed with severe GVHD (cutaneous stage 4, overall grade IV; unresolved; mortality day 91; FIG. 3D). cfDNA tissue-of-origin profiling for samples after diagnosis of this patient revealed an increase in solid-organ derived cfDNA in the blood of this patient despite increasingly potent GVHD treatment (233.8 ng/mL at month 1 and 1217.7 ng/mL at month 2; tacrolimus at month 1, and tacrolimus, sirolimus, ruxolitinib, and glucocorticoids at month 2). These three examples illustrate the potential utility of cfDNA tissue-of-origin profiling to monitor GVHD treatment response and outcome.

### Plasma Infectome after HCT

It is not only human host cells that shed their DNA into the blood; cfDNA from viruses and bacteria can be detected in the circulation, providing a means to screen for infection via metagenomic cfDNA sequencing. This may be a particularly powerful approach in the context of HCT, given the high incidence of infectious complications, and the broad range of microorganisms that can cause disease in HCT. To test this concept, the inventors mined all data from all patients for microbial derived sequences. To identify microbial-derived cfDNA after WGBS, the inventors first identified and removed host related sequences and the inventors then aligned the remaining unmapped reads to a set of microbial reference genomes (0.9±0.4% of total reads, Materials and Methods). The inventors implemented a background correction algorithm to remove contributions due to alignment noise and environmental contamination.

Using this procedure, the inventors found a significant increase in the burden of cfDNA derived from DNA viruses after HCT (viral cfDNA biomass 1.4×10⁻³ ng/mL and 1.5×10⁻² ng/mL, day of transplant and at month 3 respectively, p-value =0.0082, FIG. 4A), but not in bacterial cfDNA (bacterial cfDNA biomass 9.8×10⁻³ ng/mL and 3.3×10⁻² ng/mL, day of transplant and at month 3 respectively, p-value=0.50). The inventors have discovered a link between the abundance in plasma of Anelloviridae and the degree of immunosuppression in solid-organ transplantation, and HCT. In line with these observations, the increase in cfDNA derived from DNA viruses was largely due to an increase in the burden of Anelloviridae cfDNA in the first months after HCT (FIG. 4B). Herpesviridae and Polyomaviridae frequently establish latent infection in adults and may reactivate after allogeneic HCT. The inventors identified cfDNA from Human Herpesviridae and Polyomaviridae in 35 of 106 samples from 15 of 18 patients (FIG. 4C). In contrast to Anelloviridae, the inventors did not observe a consistent increase in the burden of cfDNA from these viruses after HCT (FIG. 4B). The detection of BK polyomavirus is concordant with clinical diagnosis of BK virus disease. Four individuals in the cohort were diagnosed with BK polyomavirus disease, defined here as the combination of clinical BK polyomavirus disease symptoms and a positive blood or urine BK PCR test in the absence of other causes of genitourinary pathology (see Methods). The instant metagenomic assay detected BK polyomavirus cfDNA in the plasma of 4 out of 5 patients with a positive BK blood test (Spearman's rho 0.79, p-value<2.2×10⁻¹⁶), but did not for patients with a negative blood test but a positive urine test, (n=15).

The inventors identified cfDNA from 7 different genera of bacteria (10 species). Interestingly, all of the identified species are well documented intestinal commensal organisms, in agreement with a loss of the integrity of the gut vascular barrier associated with GVHD. For a single patient with unresolved stage IV skin GVHD, the inventors identified a potential bloodstream infection with Klebsiella pneumoniae. Two patients in this cohort developed a clinically diagnosed Streptococcus bloodstream infection within the first 6 months post-transplant. The inventors did not detect Streptococcus cfDNA by metagenomic cfDNA sequencing for these two patients, potentially because the infection timepoints were at least six days away from the nearest plasma collection time point and bloodstream infections with Streptococcus species rapidly clear after the initiation of antimicrobial treatment.

The inventors have herein described a cfDNA assay with the potential to detect both GVHD related injury and infection after allogeneic HCT. The inventors reasoned that cfDNA may also inform injury to vascularized tissues due to GVHD after HCT. To quantify cfDNA derived from any tissue, the inventors implemented bisulfite sequencing of cfDNA, to profile cytosine methylation marks that are comprised within cfDNA and that are cell, tissue and organ type specific. Several other epigenetic marks, including hydroxymethylation and histone modifications, can inform the tissues-of-origin of cfDNA, and profiling of these marks may also be useful to monitor GVHD after HCT.

In recent years, several protein biomarkers have been investigated for the diagnosis of GVHD. Proteomic approaches have yielded candidate markers that are not directly involved in the pathogenesis of GVHD, but that are secreted as a result of end-organ damage. ST2 and REG3a, which both derive from the gastrointestinal tract, are two such biomarkers with the strongest predictive power. The cfDNA assay presented here may provide inherent advantages over protein biomarker technologies. First, because the concentration of tissue-specific DNA can be directly related to the degree of cellular injury, this assay is easy to interpret, and offers a measure of injury that can in principle be followed over time. Second, the cfDNA assay explored here provides a generalizable approach to measure injury to any tissue, whereas protein injury markers may not be available for all cell and tissue types. Third, this assay is compatible with a variety of quantitative nucleic acid measurement technologies, including digital and quantitative PCR and DNA sequencing. Fourth, this assay does not depend on antibodies, which come with challenges of specificity and reproducibility.

Whole genome bisulfite sequencing is not only responsive to human host derived cfDNA, but also to microbial cfDNA that may be present in the blood circulation. The inventors investigated the potential to screen for microbial and viral derived cfDNA in plasma of HCT recipients via bisulfite sequencing of cfDNA. BK virus cfDNA was detected using this approach for samples that were BK virus positive in blood, but not for those that were only BK virus positive in urine. In addition, while the inventors note increased herpesvirus genomic abundance, during immunosuppression, differentiating lytic and latent infection and the corresponding host response could be vital in improving patient care. The assay reported here therefore has the potential to simultaneously inform about GVHD, from the tissues-of-origin of host cfDNA, and infection, from metagenomic analysis of microbial cfDNA. Compared to conventional metagenomic sequencing, this assay requires one additional experimental step to bisulfite convert cfDNA, which can be completed within approximately 2 hours and is compatible with multiple existing next-generation sequencing workflows.

### Example 3

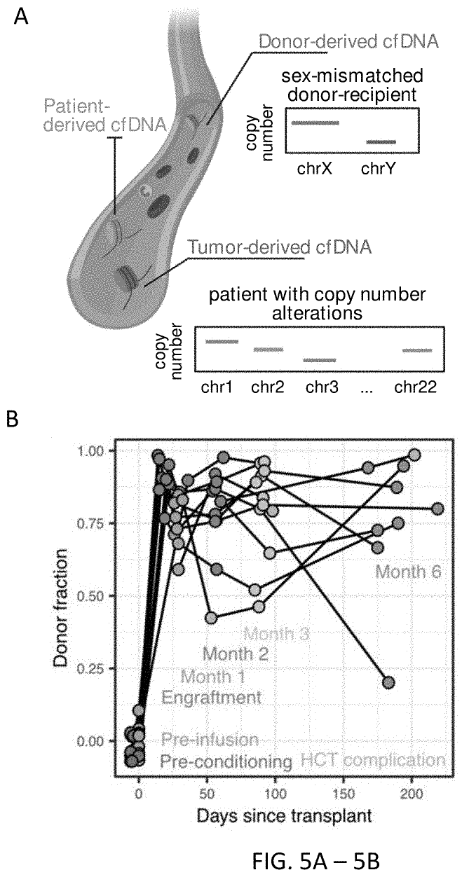

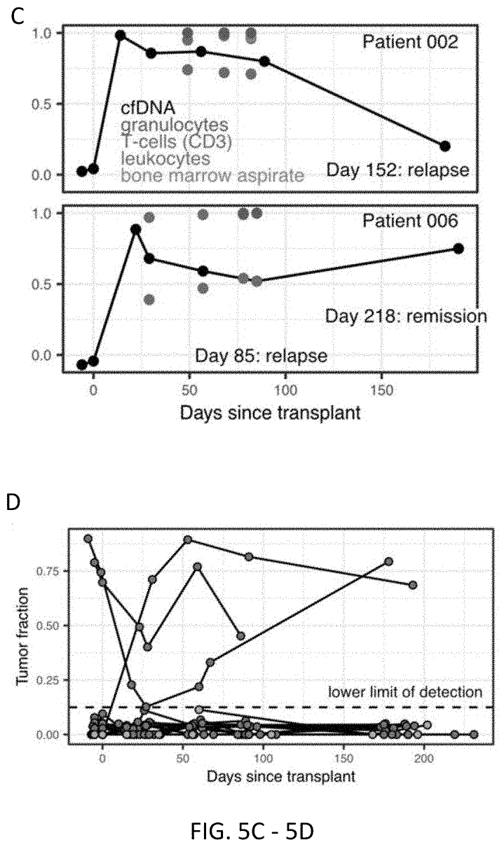

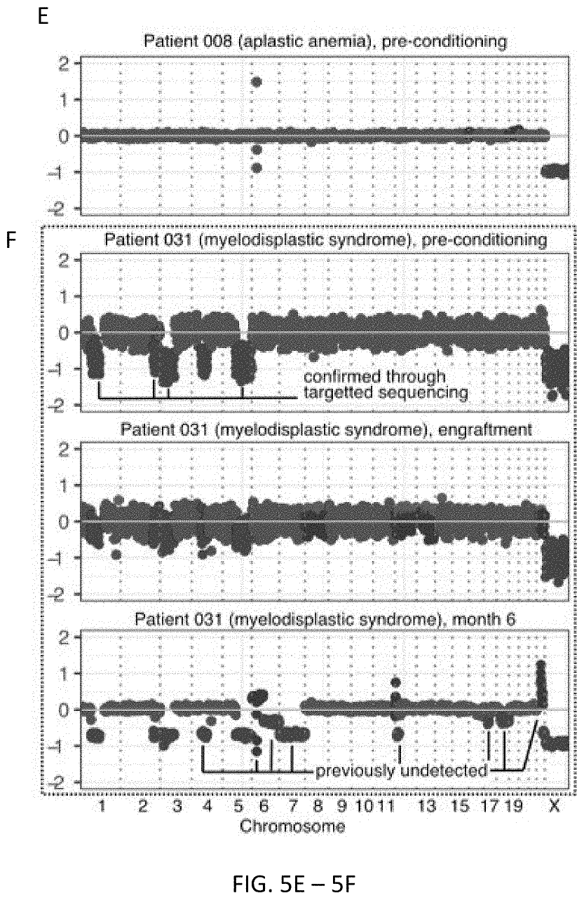

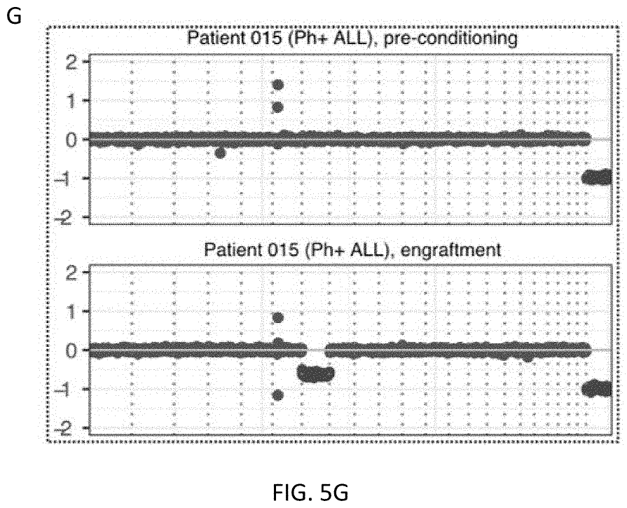

Schematic of potential sources of cell-free DNA (cfDNA) in blood is found in FIG. 5A. cfDNA can come from a Hematopoietic cell transplant (HCT) donor, an HCT recipient's (i.e., the patient's) own non-tumor tissues, a tumor tissue, or a microbial infection. In all cases of allogeneic HCT, one can measure a cfDNA donor fraction. Generally, this can be done by identifying genetic differences in the HCT patient and the donor, and looking for those differences in cfDNA molecules. In sex-mismatched donor-recipients, counting the number of cfDNA fragments from the X and Y chromosomes easily provides the donor fraction (FIG. 5A, top inlet). In the case of certain malignant blood disorders, tumor-derived cfDNA can be detected through genetic changes that occur only in the tumor. For example, this can arise as single-nucleotide polymorphisms, or copy-number changes (loss or gain of chromosomes, or loss or gains of parts of chromosomes) (FIG. 5A, lower inlet).

### Donor Fraction Measurements for Patients Over Time

FIGS. 5B-5C show donor fraction measurements from sex-mismatched patients. Donor fractions are 0 before the transplant (as there is no donor-derived DNA, but is quite elevated at Engraftment (when there are clinical signs that the graft is producing a certain amount of blood cells)). FIG. 5C provides two examples where patients experienced relapse (recurrence of blood disorder) that could be picked up via the donor fraction measurements. When there is relapse, the donor fraction decreases, and when there is remission, the donor fraction increases.

### Using Copy Number Alterations (CNAs) to Estimate Tumor Fraction

FIG. 5D demonstrates that copy number alterations can be used to estimate a tumor fraction in cell-free DNA. Not all patients had malignant blood disorders with measurable copy-number alterations. However, when a tumor has copy number alterations, those alterations can be used to monitor the presence and progression of that tumor.

### Genome-Wide Coverage Plots of cfDNAs can Uncover CNAs

FIGS. 5E-5F shows genome-wide coverage plots made by mapping the detected cfDNA fragments to the human genome. Patient 008 has a nonmalignant blood disorder which does not have any copy number alterations (FIG. 5E). Patient 031, on the other hand, is an HCT patient (FIG. 5F). Patient 031 is shown at three different time points: Pre-conditioning: prior to receiving HCT, Engraftment, and 6 months after engraftment. Pre-conditioning: prior to receiving their transplant, this individual had 5 copy number alterations (The copy number alteration on the X chromosome does not count, as this patient was a male, having one X and one Y chromosomes, instead of 2X chromosomes). The inventors also performed a clinical test (Rapid Heme Panel) as a comparison before the transplant, and the method described herein identified the same copy number alterations and more. The current cfDNA-based method is superior because it profiles the entire genome, whereas Rapid Heme Panel only profiles 95 genes. The instant cfDNA assay, which was used to monitor patients throughout their transplant, captured the development of new copy number alterations in patient 031. By month 6, the patient had multiple, previously undetected, copy number alterations. This may suggest the development of a new cancer, or the selection of a subclonal population.

The methodology described herein can also be used to track genomic changes in patients over time. FIG. 5G shows an example of a patient who had no copy number alterations at baseline, but suddenly lost one copy of chromosome 7.

What is claimed is:

1. A method for detecting tissue damage in a subject comprising:
obtaining cfDNA molecules from a biological sample from the subject;
determining the profiles of an epigenetic marker within the cfDNA molecules, wherein the epigenetic marker displays tissue-specific profiles;
identifying the tissues of origin of the cfDNA molecules based on the profiles determined; and
measuring the level of cfDNA molecules from an identified tissue of origin, wherein (i) the level or (ii) an increased level of cfDNA molecules from said identified tissue of origin as compared to a control level, is indicative of damage in said identified tissue of origin.

2. The method of claim 1, wherein the epigenetic marker is selected from the group consisting of a DNA modification, a histone modification, and nucleosome positioning.

3. The method of claim 2, wherein the DNA modification is DNA methylation or DNA hydroxymethylation.

4. The method of claim 2, wherein the histone modification is selected from the group consisting of acetylation, methylation, phosphorylation, ubiquitylation, GlcNAcylation, citrullination, krotonilation, and isomerization.

5. The method of claim 2, wherein the determining the profiles of the epigenetic marker comprises determining the sequences of the cfDNA molecules.

6. The method of claim 3, wherein the profile of DNA methylation is determined by bisulfite treatment or enzymatic DNA methylation analysis.

7. The method of claim 3, wherein the profile of DNA hydroxymethylation is determined by a pull-down assay, a selective labeling assay, or an oxidative bisulfite sequencing assay.

8. The method of claim 4, wherein the profile of histone modification is detected by a pull-down assay.

9. The method of claim 2, wherein the nucleosome positioning is determined by a nucleosome positioning assay.

10. The method of claim 2, wherein the determining the profiles of the epigenetic marker is achieved without determining the sequences of the cfDNA molecules.

11. The method of claim 10, wherein the determining is achieved by a PCR assay selected from quantitative PCR (qPCR) and digital droplet PCR (ddPCR).

12. The method of claim 11, wherein the assay comprises amplifying cfDNA molecules from regions of the genome that have specific epigenetic markers.

13. The method of claim 1, wherein, prior to determining, a library of cfDNA molecules is prepared using single-stranded DNA (ssDNA) library preparation method.

14. The method of claim 1, wherein the subject has undergone hematopoietic cell transplantation (HCT).

15. The method of claim 14, wherein the biological sample is a blood or a serum sample.

16. The method of claim 14, wherein the biological sample is obtained from the subject about 15 days, about 30 days, about 45 days, about 60 days, or about 90 days after the HCT.

17. The method of claim 14, wherein the control level is (i) the level of cfDNA molecules in a sample from the subject prior to HCT, or (ii) the level of cfDNA molecules in a sample from a subject who has undergone HCT but who has not had graft-versus-host disease (GVHD).

18. The method of claim 1, wherein the tissues of origin comprise a solid organ.

19. The method of claim 18, wherein the solid organ is an organ selected from kidney, liver, spleen, and pancreas.

20. The method of claim 1, wherein the tissues of origin comprise a tumor.

21. The method of claim 1, wherein the cfDNA molecules are from one or more organs selected from skin, heart, kidney, liver, lungs, stomach, bladder or pancreas.

22. The method of claim 1, wherein when there is a tissue damage in the subject, the method further comprises treating the subject with a therapy to ameliorate a damaged tissue in the subject.

23. The method of claim 22, wherein the therapy comprises administration of an immunoregulatory agent to the subject.

24. The method of claim 23, wherein the damaged tissue is selected from skin, heart, kidney, liver, lungs, stomach, small intestine, large intestine, bladder, and pancreas, and the immunoregulatory agent is an anti-inflammatory drug, a steroid, an antibody, or a small molecule drug.

25. The method of claim 1, wherein detecting tissue damage at about 30 days post-HCT is indicative of organ rejection or a risk of developing organ rejection.

26. The method of claim 1, wherein the tissue damage is indicative of graft-versus-host disease (GVHD).

27. The method of claim 26, further comprising treating the subject with an immunoregulatory agent when there is GVHD in the subject.

28. The method of claim 1, wherein the tissue damage is indicative of a microbial infection.

29. The method of claim 28, further comprising treating the subject with an antibiotic or an antiviral drug.

30. The method of claim 1, wherein the tissue damage is indicative of drug toxicity.

31. A method for monitoring a subject who has undergone hematopoietic cell transplantation (HCT) comprising:
obtaining cfDNA molecules from a biological sample from the subject;
determining the profiles of an epigenetic marker within the cfDNA molecules, wherein the epigenetic marker displays tissue-specific profiles;
identifying the tissues of origin of the cfDNA molecules based on the profiles determined; and
measuring the level of cfDNA molecules from an identified tissue of origin, wherein an increased level of cfDNA molecules from said identified tissue of origin as compared to a control level is indicative of graft-versus-host disease.

32. The method of claim 31, wherein the epigenetic marker is selected from the group consisting of a DNA modification, a histone modification, and nucleosome positioning.

33. The method of claim 32, wherein the DNA modification is DNA methylation or DNA hydroxymethylation.

34. The method of claim 32, wherein the histone modification is selected from the group consisting of acetylation, methylation, phosphorylation, ubiquitylation, GlcNAcylation, citrullination, krotonilation, and isomerization.

35. The method of claim 32, wherein the determining the profiles of the epigenetic marker comprises determining the sequences of the cfDNA molecules.

36. The method of claim 33, wherein the profile of DNA methylation is determined by bisulfite treatment or enzymatic DNA methylation analysis.

37. The method of claim 33, wherein the profile of DNA hydroxymethylation is determined by a pull down assay, a selective labeling assay, or an oxidative bisulfite sequencing assay.

38. The method of claim 32, wherein the profile of histone modification is detected by a pull-down assay.

39. The method of claim 32, wherein the nucleosome positioning is determined by a nucleosome positioning assay.

40. The method of claim 31, wherein, prior to determining, a library of cfDNA molecules is prepared using single-stranded DNA (ssDNA) library preparation method.

41. The method of claim 32, wherein the determining the profiles of the epigenetic marker is achieved without determining the sequences of the cfDNA molecules.

42. The method of claim 41, wherein the determining is achieved by a PCR assay selected from quantitative PCR (qPCR) and digital droplet PCR (ddPCR).

43. The method of claim 41, wherein the assay comprises amplifying cfDNA molecules from regions of the genome that have specific epigenetic markers.

44. The method of claim 31, wherein the biological sample is a blood, a plasma or a serum sample.

45. The method of claim 31, wherein the biological sample is obtained from the subject about 15 days, about 30 days, about 45 days, about 60 days, about 75 days, about 90 days, about 105 days, or about 120 days after the HCT.

46. The method of claim 31, wherein the control level is (i) the level of cfDNA molecules in a sample from the subject prior to HCT, or (ii) the level of cfDNA in a sample from a subject who has undergone HCT but who has not had graft-versus-host disease (GVHD).

47. The method of claim 31, wherein the tissues of origin comprise a solid organ.

48. The method of claim 47, wherein the solid organ comprises an organ selected from kidney, liver, spleen, pancreas.

49. The method of claim 31, wherein the cfDNA molecules are from an organ selected from skin, heart, kidney, liver, lungs, stomach, bladder and pancreas.

50. The method of claim 31, further comprising treating the subject with an immunoregulatory agent when there is graft-versus-host disease in the subject.

51. The method of claim 31, wherein the biological sample is obtained from the subject at about 30 days post-HCT.

52. A method for detecting microbial infection in a biological sample from a subject comprising:
obtaining cell-free DNA (cfDNA) molecules from the biological sample;
determining the sequences of the cfDNA molecules; and
identifying the presence of a cfDNA sequence of a microbial species, thereby detecting an infection by the microbial species.

53. The method of claim 52, wherein, prior to determining, a library of cfDNA molecules is prepared using single-stranded DNA (ssDNA) library preparation method.

54. The method of claim 52, wherein the cfDNA molecules are bisulfite treated before determining the sequences of the cfDNA molecules.

55. The method of claim 52, further comprising treating the subject with an anti-microbial agent when a microbial cfDNA sequence is identified in the biological sample.

56. The method of claim 55, wherein the anti-microbial agent is an anti-bacterial or anti-fungal agent.

57. The method of claim 55, wherein the anti-microbial agent is an anti-viral agent.

58. The method of claim 52, wherein the subject has undergone hematopoietic cell transplantation (HCT).

59. The method of claim 58, wherein the biological sample is a blood or a serum sample.

60. A method comprising:
obtaining cfDNA molecules from a biological sample from a subject;
determining the profiles of an epigenetic marker within the cfDNA molecules, wherein the epigenetic marker displays tissue-specific profiles;
identifying the tissues of origin of the cfDNA molecules based on the profiles determined;
measuring the level of cfDNA molecules from an identified tissue of origin, wherein an increased level of cfDNA molecules from said identified tissue of origin as compared to a control level is indicative of damage in said identified tissue of origin; and
identifying the presence of a microbial cfDNA in the biological sample.

61. The method of claim 60, where the biological sample has been bisulfite treated.

62. The method of claim 60, wherein the identifying the presence of a microbial cfDNA in the biological sample comprises determining the sequences of the cfDNA molecules.

63. The method of claim 60, wherein the subject has undergone hematopoietic cell transplantation (HCT).

64. A method for detecting a tumor in a subject comprising:
obtaining cell-free DNA (cfDNA) molecules from a biological sample from the subject;
identifying the presence of a tumor-derived cfDNA molecule based on a tumor-specific DNA alteration; and
measuring the level of the tumor-derived cfDNA molecule, wherein an increased level of tumor-derived cfDNA molecule as compared to a control level is indicative of the presence of tumor in the subject, or wherein an increased level of tumor-derived cfDNA molecule as compared to a level at an earlier time in the subject is indicative of tumor progression in the subject.

65. The method of claim 64, wherein, prior to determining, a library of cfDNA molecules is prepared using single-stranded DNA (ssDNA) library preparation method.

66. The method of claim 65, wherein the tumor-specific DNA alteration is selected from a tumor-specific deletion, a tumor-specific amplification, or a tumor-specific point mutation.

67. The method of claim 65, wherein the cfDNA molecules are bisulfite treated.

68. The method of claim 67, wherein the tumor-specific DNA alteration is tumor-specific DNA methylation.

69. The method of claim 64, wherein when an increased level of tumor-derived cfDNA molecules is detected, the method further comprises treating the subject with chemotherapy, a radiotherapy, or a combination therapy.

70. The method of claim 69, wherein the chemotherapy is selected from a DNA alkylating agent, an antimetabolite, an anti-tumor antibiotic, a topoisomerase inhibitor, a mitotic inhibitor, or a corticosteroid.

71. The method of claim 64, wherein the subject has undergone hematopoietic cell transplantation (HCT).

72. The method of claim 64, wherein the biological sample is a blood or a serum sample.

73. A method for monitoring engraftment a subject who has undergone hematopoietic cell transplantation (HCT) from a donor comprising:
obtaining cfDNA molecules from a biological sample from the subject;
determining the profiles of a marker within the cfDNA molecules, wherein the marker has different profiles between the subject and the donor;
identifying the origin of the cfDNA molecules based on the profiles determined; and
measuring the level of cfDNA molecules from the subject and the level of cfDNA molecules from the donor, wherein an increased ratio of cfDNA molecules from the subject versus cfDNA molecules from the donor as compared to a control ratio is indicative of loss of engraftment.

74. The method of claim 73, wherein, prior to determining, a library of cfDNA molecules is prepared using single-stranded DNA (ssDNA) library preparation method.

75. The method of claim 74, wherein the marker is selected from the group consisting of a sex chromosome, a DNA modification, a histone modification, and nucleosome positioning.

76. The method of claim 75, wherein the DNA modification is DNA methylation or DNA hydroxymethylation.

77. The method of claim 75, wherein the histone modification is selected from the group consisting of acetylation, methylation, phosphorylation, ubiquitylation, GlcNAcylation, citrullination, krotonilation, and isomerization.

78. The method of claim 75, wherein the determining the profiles of the epigenetic marker comprises determining the sequences of the cfDNA molecules.

79. The method of claim 76, wherein the profile of DNA methylation is determined by bisulfite treatment or enzymatic DNA methylation analysis.

80. The method of claim 76, wherein the profile of DNA hydroxymethylation is determined by a pull down assay, a selective labeling assay, or an oxidative bisulfite sequencing assay.

81. The method of claim 77, wherein the profile of histone modification is detected by a pull-down assay.

82. The method of claim 75, wherein the nucleosome positioning is determined by a nucleosome positioning assay.

83. The method of claim 75, wherein the determining the profiles of the epigenetic marker is achieved without determining the sequences of the cfDNA molecules.

84. The method of claim 83, wherein the determining is achieved by a PCR assay selected from quantitative PCR (qPCR) and digital droplet PCR (ddPCR).

85. The method of claim 84, wherein the assay comprises amplifying cfDNA molecules from regions of the genome that have specific epigenetic markers.

86. The method of claim 74, wherein the biological sample is a blood, a plasma or a serum sample.

87. The method of claim 74, wherein the biological sample is obtained from the subject about 15 days, about 30 days, about 45 days, about 60 days, about 75 days, about 90 days, about 105 days, or about 120 days after the HCT.

88. The method of claim 74, wherein the control level is (i) the level of cfDNA molecules in a sample from the subject prior to HCT, or (ii) the level of cfDNA in a sample from a subject who has undergone HCT but who has not had loss of engraftment.

89. The method of claim 74, further comprising treating the subject with an immunoregulatory agent when there is loss of engraftment.

90. The method of claim 74, wherein the biological sample is obtained from the subject at about 30 days post-HCT.
